(19) United States
(12) Patent Application Publication
(10) Pub. No.: US 2022/0415520 A1
(43) Pub. Date: Dec. 29, 2022

(54) SYSTEMS AND METHODS OF ASSESSING SIGNIFICANCE OF METABOLITES IN DISEASES

(71) Applicants: Trevor Cickovski, Miami, FL (US); Giri Narasimhan, Miami, FL (US); Kalai Mathee, Miami, FL (US)

(72) Inventors: Trevor Cickovski, Miami, FL (US); Giri Narasimhan, Miami, FL (US); Kalai Mathee, Miami, FL (US)

(73) Assignee: The Florida International University Board of Trustees, Miami, FL (US)

(21) Appl. No.: 17/356,868

(22) Filed: Jun. 24, 2021

(51) Int. Cl. G16H 50/70; G16B 20/00; G16H 10/40; G16H 10/60; G16H 50/20; G06F 16/9035

(52) U.S. Cl. CPC G16H 50/70; G16B 20/00; G16H 10/40; G16H 10/60; G16H 50/20; G06F 16/9035

(57) ABSTRACT

Systems and methods of assessing significance of metabolites in diseases are provided. A multi-omics downstream analysis pipeline can represent lung microbiome ecology as a heterogeneous network of microbes and metabolites. Such an analysis can demonstrate the significance of a particular metabolite in the microbiome of a patient (e.g., a human patient).

### BACKGROUND OF THE INVENTION

Glutamine is an amino acid that is produced naturally by the body, though it can also be obtained through food or supplements. Glutamine is known to be important for proper immune and intestinal function, and in the lung its depletion has been correlated with acute respiratory distress syndrome (ARDS) and asthma. Also, connections between the lung microbiome and both alpha-1 antitrypsin deficiency (A1AD) and its post-cursor chronic obstructive pulmonary disease (COPD) have been established, including severity of COPD symptoms and the streptococcus genus.

### BRIEF SUMMARY

Embodiments of the subject invention provide novel and advantageous systems and methods of assessing significance of metabolites in diseases of the lungs. A multi-omics downstream analysis pipeline can represent lung microbiome ecology as a heterogeneous network of microbes and metabolites. Such an analysis can demonstrate the significance of a particular metabolite (e.g., glutamine) in the lung microbiome of a patient (e.g., a mammalian patient such as a human patient), including a patient with a lung disease (e.g., alpha-1 antitrypsin deficiency (A1AD) and/or chronic obstructive pulmonary disease (COPD)).

In an embodiment, a system for assessing significance of metabolites in a disease of the lungs can comprise a processor and a (non-transitory) machine-readable medium in operable communication with the processor and having instructions stored thereon that, when executed by the processor, perform the following steps: receiving raw metagenomics data of a plurality of patients (e.g., human patients) having the disease of the lungs; receiving raw metabolomics data of the plurality of patients having the disease of the lungs; passing the raw metagenomics data through a metagenomics pipeline to obtain raw microbial abundances; normalizing the raw microbial abundances to obtain normalized microbial abundances; normalizing the raw metabolomics data to obtain normalized metabolomics data; performing Spearman correlations on the normalized microbial abundances and the normalized metabolomics data to obtain microbe-microbe correlations, microbe-metabolite correlations, and metabolite-metabolite correlations; performing a filtering process on the microbe-microbe correlations, the microbe-metabolite correlations, and the metabolite-metabolite correlations to obtain a (heterogeneous) multi-omics co-occurrence network of microbes and metabolites associated with the disease of the lungs; and performing a centrality analysis on the multi-omics co-occurrence network to identify the significance of metabolites in the disease of the lungs. The raw metagenomics data can comprise, for example, ribonucleic acid (RNA) reads, such as 16S RNA reads. The raw metabolomics data can comprise metabolite concentrations. The filtering process can comprise performing a first filter step in which a filtering tool is used to filter the microbe-metabolite correlations according to a first filter rule and to filter the metabolite-metabolite correlations according to a second filter rule. The first filter rule can be that a correlation between a first metabolite and a second metabolite is kept if a reaction involving the first metabolite and the second metabolite exists in the filtering tool that occurs either within a patient of the plurality of patients or within at least one microbe of a set of all microbes in the microbe-microbe correlations, the microbe-metabolite correlations, and the metabolite-metabolite correlations. The second filter rule can be that a correlation between a first microbe and a third metabolite is kept if a reaction involving the third metabolite exists in the filtering tool that occurs within the first microbe. The filtering process can further comprise performing a second filter step in which the microbe-microbe correlations are filtered according to a third filter rule. The third filter rule can be that a correlation between a second microbe and a third microbe is kept if a path exists between the second microbe and the third microbe that involves no microbe-microbe edges. The second filter step can be performed after the first filter step has completed. The disease of the lungs can be, for example, A1 AD or COPD.

In another embodiment, a method for assessing significance of metabolites in a disease of the lungs can comprise: receiving (e.g., by a processor) raw metagenomics data of a plurality of patients (e.g., human patients) having the disease of the lungs; receiving (e.g., by the processor) raw metabolomics data of the plurality of patients having the disease of the lungs; passing (e.g., by the processor) the raw metagenomics data through a metagenomics pipeline to obtain raw microbial abundances; normalizing (e.g., by the processor) the raw microbial abundances to obtain normalized microbial abundances; normalizing (e.g., by the processor) the raw metabolomics data to obtain normalized metabolomics data; performing (e.g., by the processor) Spearman correlations on the normalized microbial abundances and the normalized metabolomics data to obtain microbe-microbe correlations, microbe-metabolite correlations, and metabolite-metabolite correlations; performing (e.g., by the processor) a filtering process on the microbe-microbe correlations, the microbe-metabolite correlations, and the metabolite-metabolite correlations to obtain a (heterogeneous) multi-omics co-occurrence network of microbes and metabolites associated with the disease of the lungs; and performing (e.g., by the processor) a centrality analysis on the multi-omics co-occurrence network to identify the significance of metabolites in the disease of the lungs. The raw metagenomics data can comprise, for example, RNA reads, such as 16S RNA reads. The raw metabolomics data can comprise metabolite concentrations. The filtering process can comprise performing a first filter step in which a filtering tool is used to filter the microbe-metabolite correlations according to a first filter rule and to filter the metabolite-metabolite correlations according to a second filter rule. The first filter rule can be that a correlation between a first metabolite and a second metabolite is kept if a reaction involving the first metabolite and the second metabolite exists in the filtering tool that occurs either within a patient of the plurality of patients or within at least one microbe of a set of all microbes in the microbe-microbe correlations, the microbe-metabolite correlations, and the metabolite-metabolite correlations. The second filter rule can be that a correlation between a first microbe and a third metabolite is kept if a reaction involving the third metabolite exists in the filtering tool that occurs within the first microbe. The filtering process can further comprise performing a second filter step in which the microbe-microbe correlations are filtered according to a third filter rule. The third filter rule can be that a correlation between a second microbe and a third microbe is kept if a path exists between the second microbe and the third microbe that involves no microbe-microbe edges. The second filter step can be performed after the first filter step has completed. The disease of the lungs can be, for example, A1AD or COPD.

### BRIEF DESCRIPTION OF THE DRAWINGS

FIG. 1 shows a schematic view of a model that can be used with systems and methods according to embodiments of the subject invention. The legend in FIG. 1 shows that the stars indicate initial data; ovals are for data; squares represent computation; the green ovals (“raw 16S RNA reads”, “raw microbial abundances”, “normalized microbial abundances”, and “microbe-microbe correlations”) are for metagenomics data only; the red ovals (“raw metabolite concentrations”, “normalized metabolite concentrations”, and “metabolite-metabolite correlations”) are for metabolomics data only; the orange ovals (“microbe-metabolite correlations” and “multi-omics correlation network”) are for multi-omics data; and the black oval (“ecologically important entities”) is for final results.

FIG. 2 shows a schematic view of a Spearman computation that can be used with systems and methods according to embodiments of the subject invention. The legend from FIG. 1 also applies to FIG. 2. In FIG. 2, the green ovals are “normalized microbial abundances”, “rank of each microbe in each sample”, and “microbe-microbe correlations”) the red ovals are “normalized metabolite concentrations”, “rank of each metabolite in each sample”, and “metabolite-metabolite correlations”; and the orange oval is “microbe-metabolite correlations”.

FIG. 3 shows a schematic view of a network that can be used with systems and methods according to embodiments of the subject invention. Circles represent microbes, and squares represent metabolites.

FIG. 4 shows a multi-omics co-occurrence network for alpha-1 antitrypsin deficiency (A1AD), produced according to an embodiment of the subject invention. The nodes (squares or circles) are colored by centrality value, with red (dark) being high and light purple (light) being zero or unimportant, with orange, yellow, and green being in between (orange closest to red's value and green closest to light violet's value). The circles represent microbes, and the squares represent metabolites.

FIG. 5 shows a multi-omics co-occurrence network for chronic obstructive pulmonary disease (COPD), produced according to an embodiment of the subject invention. The nodes (squares or circles) are colored by centrality value, with red (dark) being high and light purple (light) being zero or unimportant, with orange, yellow, and green being in between (orange closest to red's value and green closest to light violet's value). The circles represent microbes, and the squares represent metabolites.

### DETAILED DESCRIPTION OF THE INVENTION

Embodiments of the subject invention provide novel and advantageous systems and methods of assessing significance of metabolites in diseases of the lungs. A multi-omics downstream analysis pipeline can represent lung microbiome ecology as a heterogeneous network of microbes and metabolites. Such an analysis can demonstrate the significance of a particular metabolite (e.g., glutamine) in the lung microbiome of a patient (e.g., a mammalian patient such as a human patient), including a patient with a lung disease (e.g., alpha-1 antitrypsin deficiency (A1AD) and/or chronic obstructive pulmonary disease (COPD)). An analysis was run on patients with A1AD and COPD; a high centrality (importance) was observed for glutamate and glutamine (in that order) in patients with A1AD, while a similar centrality was observed in patients with COPD but with glutamine having a higher centrality than glutamate. A negative co-occurrence was observed with streptococcus in patients with COPD. The latter result can help develop treatments for COPD.

The significance of a chemical or metabolite in an illness has often been measured by differential abundance (in other words, how much is present in a healthy sample vs. how much is present in a diseased sample). More recently, reaction databases provide an idea of a metabolite's role in internal reactions of a host. Embodiments of the subject invention view the effects of glutamine on the larger system ecology by analyzing its co-occurrence with other metabolites as well as members of the lung microbiome. Co-occurrences are an estimator for relationships (i.e., cooperative/competitive) between entities of a microbiome, and this ecology can be represented as a microbial co-occurrence network (MCN) with nodes representing microbes and edges representing co-occurrences (see also Faust et al., Microbial Co-occurrence Relationships in the Human Microbiome, PLOS Comp Biol 2012; 8:e1002606; which is hereby incorporated by reference in its entirety). The systems and methods of embodiments of the subject invention can extend these networks to be heterogeneous in nature, with nodes able to represent microbes or metabolites. Through the application of a centrality algorithm on heterogeneous networks built from lung microbiome data of A1AD and COPD patients, the increasingly significant role played by glutamine can be seen in the respective patient microbiomes. The importance of glutamine can therefore be measured in terms of its effect on the macroscale behavior of the ecosystem.

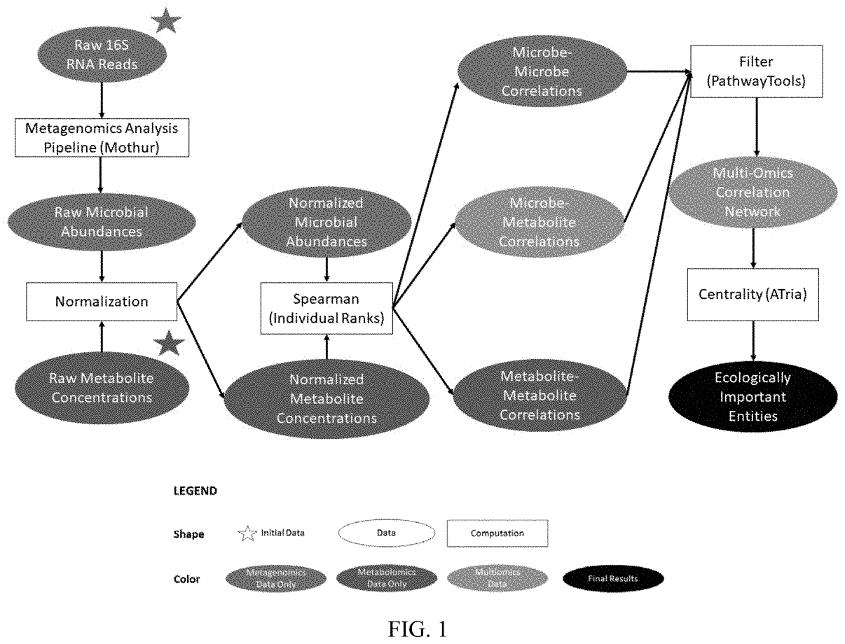

FIG. 1 shows a schematic view of a pipeline/model/algorithm that can be used with systems and methods according to embodiments of the subject invention. Referring to FIG. 1, metagenomics data (e.g., raw metagenomics data) and metabolomics data (e.g., raw metabolomics data) can be received. The metagenomics data can be, for example, ribonucleic acid (RNA) data, such as 16S RNA data. The metabolomics data can be, for example, metabolite concentrations. The metagenomics data can be passed through a standard metagenomics pipeline, such as one implemented and executed using Mothur (Schloss et al., Introducing mothur: Open-Source, Platform-Independent, Community-Supported Software for Describing and Comparing Microbial Communities, AEM 2009; 75: 7537-7541; which is hereby incorporated by reference herein in its entirety). The pipeline can denoise, cluster, and/or look up the reads in a database, producing for each sample a set of raw abundances of the microbial taxa present in the sample (“raw microbial abundances”). These abundances can then be normalized for each sample, producing a set of normalized microbial abundances. The metabolomics data (e.g., metabolite concentrations) can also be normalized for each sample, producing a set of normalized metabolite concentrations.

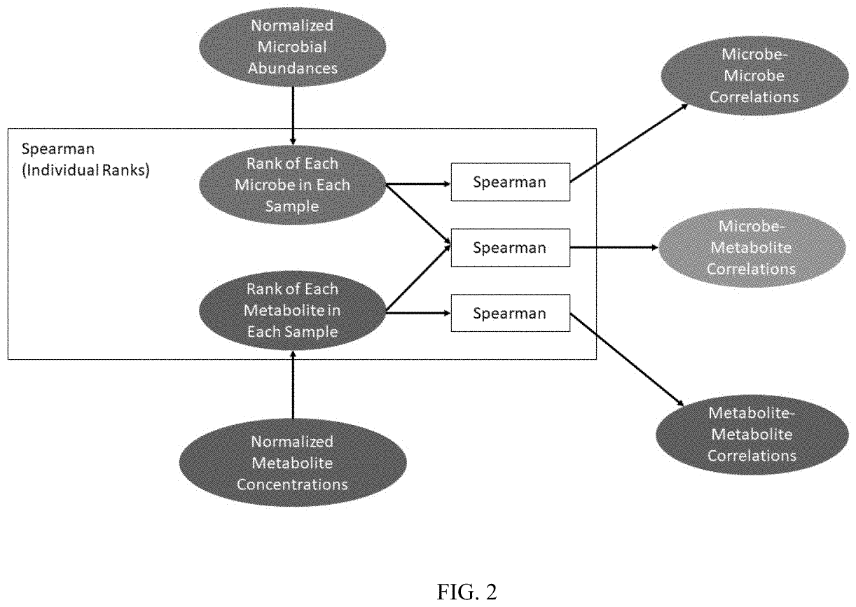

Co-occurrence networks can be used to estimate ecological relationships between entities (microbes) within microbiomes. While traditionally homogeneous in nature (i.e., only containing microbes), embodiments of the subject invention can build a heterogeneous network including microbes and metabolites. Spearman correlations can be used to form such a network (see also, Spearman, General Intelligence, Objectively Determined and Measured, The American Journal of Psychology, 1904; 15: 201; which is hereby incorporated by reference herein in its entirety). Spearman correlations operate by looking at the rank of each observable within each sample. In this case, the two sets of observables are normalized microbial abundances and normalized metabolite concentrations. The units of these two datasets are different, resulting in a different range of values, making it impossible to rank them with respect to each other. FIG. 2 shows how the Spearman computation proceeds.

In other words, each microbe has a rank in every sample by its abundance with respect to other microbes, and each metabolite has a rank in every sample by its concentration with respect to other metabolites. In order to address this, three separate iterations of Spearman correlations can be performed: one using only the microbial ranks; one using only the metabolic ranks; and one using both sets of ranks (there would therefore be two #1s, two #2s, etc.). The correlation would work the same, noting which microbes and metabolites tended to rise or fall in ranking simultaneously or inversely.

A filtering process can be included, in which correlations are filtered based on biological relevance, defined by the presence of a documented biochemical reaction in a reaction database. For example, PathwayTools from the MetaCyc toolkit can be used, though embodiments are not limited thereto (see Karp et al., The Pathway Tools software, Bioinformatics, 2002; 18: S225—S232). Being a reaction database, PathwayTools has two key pieces of data for the application: reactions involving metabolites; and organisms (including humans and microbes) in which they occur.

In a first filter step (e.g., PathwayTools Queries), the collective system can include a set of microbes X and a set of metabolites Y. This filtering proceeds as a two-step process, where a filtering tool (e.g., PathwayTools) can be used in the first step to filter metabolite-metabolite correlations and metabolite-microbe correlations, with the following two rules. The first rule is that a correlation between metabolite yᵢ and metabolite yⱼ is kept if a reaction involving yᵢ and yⱼ can be found in the filtering tool (e.g., PathwayTools) that occurs within either the human or some microbe in the set of microbes X. The second rule is that a correlation between microbe xᵢ and metabolite yⱼ is kept if a reaction involving yⱼ can be found in the filtering tool (e.g., PathwayTools) that occurs within xᵢ. In summary, for a correlation to be kept that involves a metabolite, there should be support (which can be thought of as supporting “documentation” (e.g., PathwayTools)) for this correlation, through an appropriate chemical reaction within the appropriate organism (microbe or human). This forms the initial co-occurrence network, which includes microbe-microbe (not yet filtered), microbe-metabolite (filtered), and metabolite-metabolite (filtered) correlations. A second filter step can filter microbe-microbe correlations.

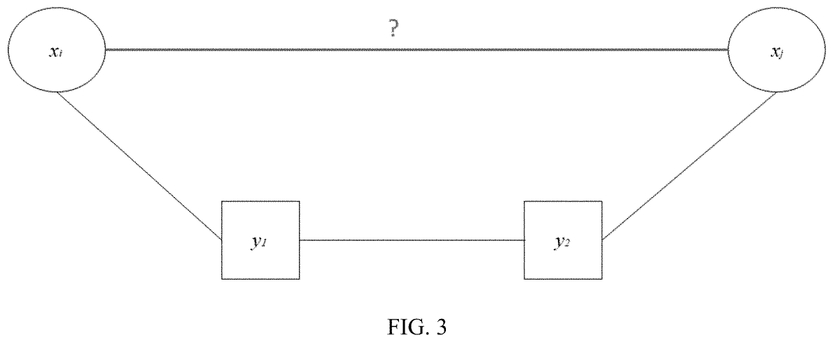

In a second filter step, queries to the filtering tool (e.g., PathwayTools) cannot be used as direct support for microbe-microbe correlations, because the database is centered around metabolomics data. However, all edges in the network at this point involving metabolites already have been filtered through the filtering tool (e.g., PathwayTools). Therefore, microbe-microbe correlations can be filtered using a third rule that a correlation between microbe xᵢ and microbe xⱼ is kept if a path can be found between them in the network that involves no microbe-microbe edges. This can be seen in the example in FIG. 3, in which circles represent microbes and squares represent metabolites. Microbe-microbe correlation xᵢ-xⱼ (the edge with the question mark in FIG. 3) is the edge under consideration as to whether to keep it or filter it out. Because all edges involving metabolites (y) have been filtered already in the first filter step, edge xᵢy₁ is evidence that there is some reaction (e.g., a filtering tool reaction (e.g., PathwayTools reaction)) occurring in microbe xᵢ that involves metabolite y₁. Edge y₁-y₂ is evidence that there is some reaction (e.g., a filtering tool reaction (e.g., PathwayTools reaction)) involving metabolites y₁ and y₂ in either a microbe or the host in the ecosystem; and edge xⱼ-y₂ is evidence that there is some reaction (e.g., a filtering tool reaction (e.g., PathwayTools reaction)) occurring in microbe xⱼ that involves metabolite y₂. Thus, because microbes xᵢ and xⱼ are connected by some documented reaction(s) (e.g., filtering tool reaction(s) (e.g., PathwayTools reaction(s))), edge xᵢ-xⱼ is kept. If such a path (again, only involving metabolites) did not exist, the edge would be discarded. The second (final) filtering step produces the final multi-omics co-occurrence network.

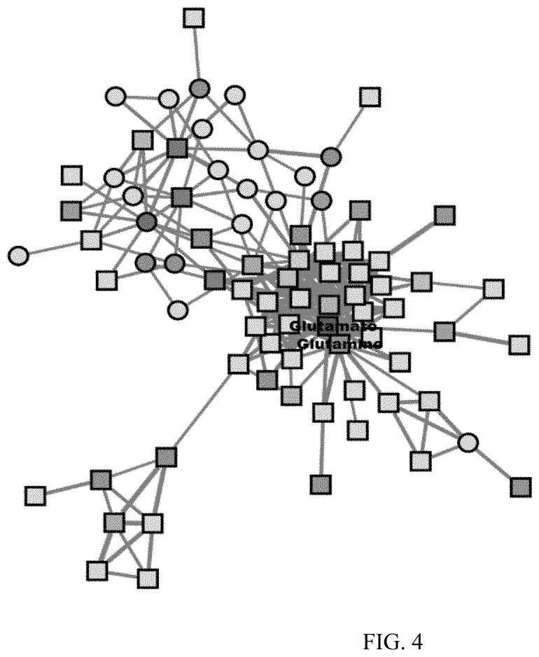

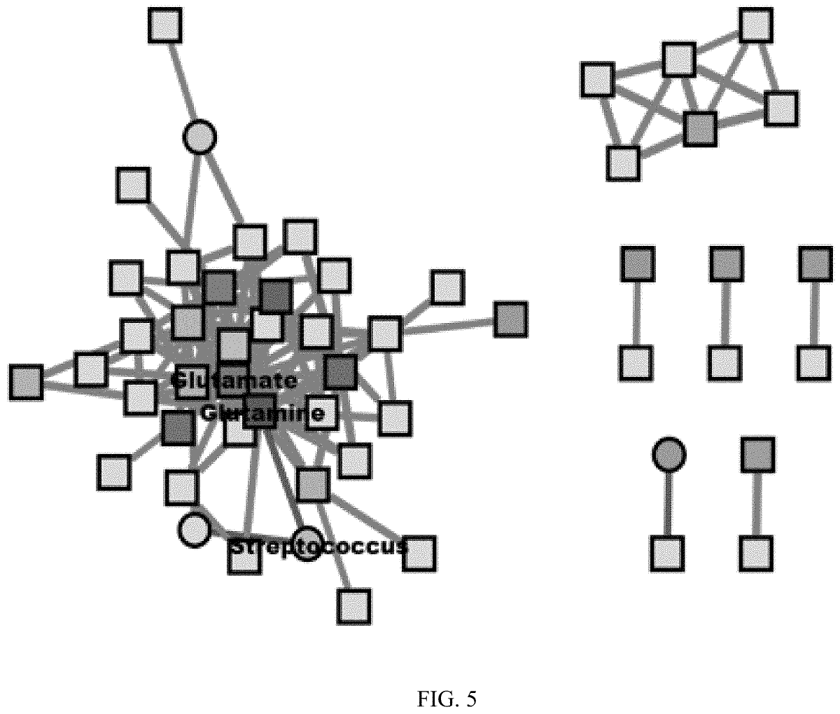

In a further embodiment, a module or algorithm (e.g., Ablatio Triadum (ATria)) can be run to find the most important or central nodes within a multi-omics co-occurrence network (Cickovski et al., ATria: A novel centrality algorithm applied to biological networks, BMC Bioinformatics 2017; 18: 239; which is hereby incorporated by reference herein in its entirety). FIGS. 4 and 5 show results of running such an algorithm (ATria) for A1AD and COPD, respectively. Circles in the networks of FIGS. 4 and 5 represent microbes, and squares represent metabolites. The nodes are colored by centrality value (red=high, orange=next highest, yellow=next highest, green=next highest, and light violet=zero or unimportant).

The size and complexity of the interaction web involving lung microbes and metabolites, coupled with the fact that everyone has a unique lung microbiome and metabolome, makes developing treatments for any lung disorder challenging. Guidance that involves recommended target microbes and metabolites for treatment is highly essential, and can potentially save a great deal of experimental costs. In addition to showing that glutamine belongs in the discussion of any candidate treatment for either A1AD or COPD, the co-occurrence networks of embodiments of the subject invention can also provide estimates of treatment side effects on the rest of the ecosystem. In addition to opening an important door for the treatment of A1AD and COPD, there may also be implications for cancer treatment. Both of these disorders have connections with lung cancer, which has and continues to dominate the list of most deadly cancers. COPD shares many metabolic pathways with lung cancer and is associated with a two-fold increase in risk, and A1AD is associated with a four-fold increase risk of lung cancer even among non-smokers. Glutamine metabolism is also an important metabolic pathway in non-small cell lung cancer.

Embodiments of the subject invention address the technical problem of assessing significance of metabolites in a disease (e.g., a disease of the lungs) by providing the technical solution of normalizing raw microbial abundance data and raw metabolomics data, performing Spearman correlations on the normalized data to obtain correlations, and performing a filtering process on the correlations to obtain a multi-omics co-occurrence network.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e. the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

### Example 1

Raw 16S RNA reads and raw metabolite concentrations were obtained from datasets collected from lung samples of 26 human patients with A1AD and 11 human patients with COPD. The datasets had all identifying information (patient name, address, etc.) removed prior to being obtained. The algorithm shown in FIG. 1 was performed, with two separate executions—one for the data for the patients with A1AD and one for the data for the patients with COPD. The 16S RNA reads were passed through a standard metagenomics pipeline implemented and executed using Mothur, producing for each sample (e.g., A1AD patient sample and COPD patient sample) a set of raw abundances of the microbial taxa present in the sample. These abundances were then normalized for each sample, as were the metabolite concentrations, producing a set of normalized microbial abundances and metabolite concentrations. Spearman correlations were used (as shown in FIG. 2 and described in detail above in the discussion of FIG. 2) to build a heterogeneous network of microbes and metabolites. Correlations were then filtered based on biological relevance by performing the first and second filter steps discussed in detail herein, using PathwayTools as the filtering tool. This gave the final respective multi-omics co-occurrence networks for the A1AD patient sample and the COPD patient sample. Ablatio Triadum (ATria) was used to find the most important or central nodes within the multi-omics co-occurrence network, with the results shown in FIG. 4 (for the A1AD patient sample) and FIG. 5 (for the COPD patient sample). Referring to FIG. 4, in A1AD it was immediately noted the high centrality values of (in particular) glutamate (#1) and glutamine and their presence more towards the center of the network, indicating the general importance of the GABA cycle in many other metabolite reactions. Referring to FIG. 5, in the case of COPD the centrality shifted to glutamine (#1), likely because of the high negative co-occurrence with streptococcus (the most central microbe). In addition to providing support for the key role played by glutamine in these lung microbiomes, the results also provide support that this opposition between the most important microbe and metabolite is a key feature of the COPD lung microbiome, offering guidance for both further study in the area and directions to pursue for potential treatments.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

1. A system for assessing significance of metabolites in a disease of the lungs, the system comprising:
a plurality of tissue samples of lungs from a plurality of patients, respectively, having the disease of the lungs;
a processor; and
a non-transitory computer-readable medium in operable communication with the processor; and
a machine-readable medium in operable communication with the processor and the non-transitory computer-readable medium, the machine-readable medium having instructions stored thereon that, when executed by the processor, perform the following steps:
determining raw metagenomics data and raw metabolomics data from the plurality of lung samples;
passing the raw metagenomics data through a metagenomics pipeline to obtain raw microbial abundances, the metagenomics pipeline being an algorithm that performs at least one of denoising the raw metagenomics data, clustering the raw metagenomics data, and looking reads up in a database for the raw metagenomics data;
normalizing the raw microbial abundances to obtain normalized microbial abundances;
normalizing the raw metabolomics data to obtain normalized metabolomics data;
performing Spearman correlations on the normalized microbial abundances and the normalized metabolomics data to obtain microbe-microbe correlations, microbe-metabolite correlations, and metabolite-metabolite correlations;
generating a multi-omics co-occurrence network of microbes and metabolites associated with the disease of the lungs by performing a filtering process on the microbe-microbe correlations, the microbe-metabolite correlations, and the metabolite-metabolite correlations, the filtering process comprising filtering the microbe-microbe correlations, the microbe-metabolite correlations, and the metabolite-metabolite correlations based on biological relevance; and
performing a centrality analysis on the multi-omics co-occurrence network to identify the significance of metabolites in the disease of the lungs,
the filtering process comprising: performing a first filter step in which a filtering tool is used to filter the microbe-metabolite correlations according to a first filter rule and to filter the metabolite-metabolite correlations according to a second filter rule; and performing a second filter step in which the microbe-microbe correlations are filtered according to a third filter rule,
the filtering tool being a software program configured to filter bioinformatics data,
the second filter step being performed after the first filter step has completed,
the first filter rule being that a correlation between a first metabolite and a second metabolite is kept if a first reaction involving the first metabolite and the second metabolite exists in the filtering tool that occurs either within a patient of the plurality of patients or within at least one microbe of a set of all microbes in the microbe-microbe correlations, the microbe-metabolite correlations, and the metabolite-metabolite correlations, and the correlation between the first metabolite and the second metabolite is discarded if not such first reaction exists on the filtering tool,
the second filter rule being that a correlation between a first microbe and a third metabolite is kept if a second reaction involving the third metabolite exists in the filtering tool that occurs within the first microbe, and the correlation between the first microbe and the third metabolite is discarded if no such second reaction exists in the filtering tool,
the third filter rule being that a correlation between a second microbe and a third microbe is kept if a first path exists between the second microbe and the third microbe that involves no microbe-microbe edges representing co-occurrences, and the correlation between the second microbe and the third microbe is discarded if no such first path exists in the filtering tool,
the microbe-microbe correlations, the microbe-metabolite correlations, the metabolite-metabolite correlations, and the multi-omics co-occurrence network being stored on at least one of the non-transitory computer-readable medium and the machine-readable medium, and
the identifying of the significance of metabolites in the disease of the lungs improving an ability of the system to diagnose the disease of the lungs.

2. The system according to claim 1, the raw metagenomics data comprising ribonucleic acid (RNA) reads.

3. The system according to claim 2, the raw metagenomics data comprising 16S RNA reads.

4. The system according to claim 1, the raw metabolomics data comprising metabolite concentrations.

5-10. (canceled)

11. A method for assessing significance of metabolites in a disease of the lungs, the method comprising:
collecting a plurality of tissue samples of lung from a plurality of patients, respectively, having the disease of the lungs;
determining raw metagenomics and raw metabolomics of the plurality of lung samples;
receiving, by a processor in operable communication with a non-transitory computer-readable medium and a machine-readable medium of a system, the raw metagenomics data of the plurality of lung samples;
receiving, by the processor, the raw metabolomics data of the plurality of lung samples;
passing, by the processor, the raw metagenomics data through a metagenomics pipeline to obtain raw microbial abundances, the metagenomics pipeline being an algorithm that performs at least one of denoising the raw metagenomics data, clustering the raw metagenomics data, and looking reads up in a database for the raw metagenomics data;
normalizing, by the processor, the raw microbial abundances to obtain normalized microbial abundances;
normalizing, by the processor, the raw metabolomics data to obtain normalized metabolomics data;
performing, by the processor, Spearman correlations on the normalized microbial abundances and the normalized metabolomics data to obtain microbe-microbe correlations, microbe-metabolite correlations, and metabolite-metabolite correlations;
generating, by the processor, a multi-omics co-occurrence network of microbes and metabolites associated with the disease of the lungs by performing a filtering process on the microbe-microbe correlations, the microbe-metabolite correlations, and the metabolite-metabolite correlations, the filtering process comprising filtering the microbe-microbe correlations, the microbe-metabolite correlations, and the metabolite-metabolite correlations based on biological relevance; and
performing, by the processor, a centrality analysis on the multi-omics co-occurrence network to identify the significance of metabolites in the disease of the lungs,
the filtering process comprising: performing a first filter step in which a filtering tool is used to filter the microbe-metabolite correlations according to a first filter rule and to filter the metabolite-metabolite correlations according to a second filter rule; and performing a second filter step in which the microbe-microbe correlations are filtered according to a third filter rule,
the filtering tool being a software program configured to filter bioinformatics data,
the second filter step being performed after the first filter step has completed,
the first filter rule being that a correlation between a first metabolite and a second metabolite is kept if a first reaction involving the first metabolite and the second metabolite exists in the filtering tool that occurs either within a patient of the plurality of patients or within at least one microbe of a set of all microbes in the microbe-microbe correlations, the microbe-metabolite correlations, and the metabolite-metabolite correlations, and the correlation between the first metabolite and the second metabolite is discarded if no such first reaction exists in the filtering tool,
the second filter rule being that a correlation between a first microbe and a third metabolite is kept if a second reaction involving the third metabolite exists in the filtering tool that occurs within the first microbe, and the correlation between the first microbe and the third metabolite is discarded if no such second reaction exists in the filtering tool,
the third filter rule being that a correlation between a second microbe and a third microbe is kept if a first path exists between the second microbe and the third microbe that involves no microbe-microbe representing co-occurrences, and the correlation between the second microbe and the third microbe is discarded if no such first path exists in the filtering tool,
the microbe-microbe correlations, the microbe-metabolite correlations, the metabolite-metabolite correlations, and the multi-omics co-occurrence network being stored on at least one of the non-transitory computer-readable medium and the machine-readable medium, and
the identifying of the significance of metabolites in the disease of the lungs improving an ability of the system to diagnose the disease of the lungs.

12. The method according to claim 11, the raw metagenomics data comprising ribonucleic acid (RNA) reads.

13. The method according to claim 12, the raw metagenomics data comprising 16S RNA reads.

14. The method according to claim 11, the raw metabolomics data comprising metabolite concentrations.

15-19. (canceled)

20. A system for assessing significance of metabolites in a disease of the lungs, the system comprising:
a plurality of tissue samples of lungs from a plurality of patients, respectively, having the disease of the lungs;
a processor;
a non-transitory computer-readable medium in operable communication with the processor; and
a machine-readable medium in operable communication with the processor and the non-transitory computer-readable medium, the machine-readable medium having instructions stored thereon that, when executed by the processor, perform the following steps:
determining raw metagenomics data and raw metabolomics data from the plurality of lung samples;
passing the raw metagenomics data through a metagenomics pipeline to obtain raw microbial abundances, the metagenomics pipeline being an algorithm that performs at least one of denoising the raw metagenomics data, clustering the raw metagenomics data, and looking reads up in a database for the raw metagenomics data;
normalizing the raw microbial abundances to obtain normalized microbial abundances;
normalizing the raw metabolomics data to obtain normalized metabolomics data;
performing Spearman correlations on the normalized microbial abundances and the normalized metabolomics data to obtain microbe-microbe correlations, microbe-metabolite correlations, and metabolite-metabolite correlations;
generating a multi-omics co-occurrence network of microbes and metabolites associated with the disease of the lungs by performing a filtering process on the microbe-microbe correlations, the microbe-metabolite correlations, and the metabolite-metabolite correlations, the filtering process comprising filtering the microbe-microbe correlations, the microbe-metabolite correlations, and the metabolite-metabolite correlations based on biological relevance; and
performing a centrality analysis on the multi-omics co-occurrence network to identify the significance of metabolites in the disease of the lungs,
the raw metagenomics data comprising 16S ribonucleic acid (RNA) reads,
the raw metabolomics data comprising metabolite concentrations,
the filtering process comprising: performing a first filter step in which a filtering tool is used to filter the microbe-metabolite correlations according to a first filter rule and to filter the metabolite-metabolite correlations according to a second filter rule; and performing a second filter step in which the microbe-microbe correlations are filtered according to a third filter rule,
the filtering tool being a software program configured to filtering bioinformatics data,
the first filter rule being that a correlation between a first metabolite and a second metabolite is kept if a first reaction involving the first metabolite and the second metabolite exists in the filtering tool that occurs either within a patient of the plurality of patients or within at least one microbe of a set of all microbes in the microbe-microbe correlations, the microbe-metabolite correlations, and the metabolite-metabolite correlations, and the correlation between the first metabolite and the second metabolite is discarded if no such first reaction exists in the filtering tool
the second filter rule being that a correlation between a first microbe and a third metabolite is kept if a second reaction involving the third metabolite exists in the filtering tool that occurs within the first microbe, and the correlation between the first microbe and the third metabolite is discarded if no such second reaction exists in the filtering tool,
the third filter rule being that a correlation between a second microbe and a third microbe is kept if a first path exists between the second microbe and the third microbe that involves no microbe-microbe edges, representing co-occurrences, and the correlation between the second microbe and the third microbe id discarded if no such first path exists in the filtering tool,
the second filter step being performed after the first filter step has completed,
the disease of the lungs being alpha-1 antitrypsin deficiency (A1 AD) or chronic obstructive pulmonary disease (COPD),
the microbe-microbe correlations, the microbe-metabolite correlations, the metabolite-metabolite correlations, and the multi-omics co-occurrence network being stored on at least one of the non-transitory computer-readable medium and the machine-readable medium, and
the identifying of the significance of metabolites in the disease of the lungs improving an ability of the system to diagnose the disease of the lungs.
